(19) United States
(12) Patent Application Publication
(10) Pub. No.: US 2011/0218593 A1
(43) Pub. Date: Sep. 8, 2011

(54) SYSTEMS, DEVICES AND METHODS FOR THE TREATMENT OF TINNITUS

(75) Inventors: Jay Rubinstein, Seattle, WA (US); William Harrison, Anacortes, WA (US)

(73) Assignee: Silere Medical Technology, Inc.

(21) Appl. No.: 13/061,305

(22) PCT Filed: Sep. 3, 2009
(86) PCT No.: PCT/US09/55893

§ 371 Date: May 24, 2011

Related U.S. Application Data
(60) Provisional application No. 61/094,824, filed on Sep. 5, 2008.
(60) Provisional application No. 61/094,830, filed on Sep. 5, 2008.
(60) Provisional application No. 61/094,822, filed on Sep. 5, 2008.

(51) Int. Cl. A61F 11/04

(52) U.S. Cl. 607/57

(57) ABSTRACT

Provided herein are systems, devices and methods for stimulation of the cochlea that are sufficient to mimic or replace the spontaneous background neural activity of the cochlea thereby reducing or eliminating tinnitus.

### CROSS REFERENCE TO RELATED APPLICATIONS

This patent application claims priority to U.S. Provisional patent application Ser. Nos.: 61/094,822, titled “SYSTEMS AND METHODS FOR THE TREATMENT OF TINNITUS,” filed on Sep. 5, 2008; 61/094,824, titled “ELECTRODES FOR THE TREATMENT OF TINNITUS,” filed on Sep. 5, 2008; and 61/094,830, titled “STIMULATORS FOR THE TREATMENT OF TINNITUS,” filed on Sep. 5, 2008.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The systems, devices and methods described herein relate to the treatment of tinnitus.

### BACKGROUND OF THE INVENTION

Tinnitus is a condition that results in an auditory perception that is heard in the ears or in the head when external auditory stimulus is absent. This condition is characterized by the sensation of a ringing, crackling, buzzing, and whistling or pulsing type sound. It is a prevalent and common condition afflicting more than 50 million people in Europe and North America with additional large numbers estimated in South America, the Pacific rim countries and the rest of the world.

The severity of tinnitus ranges from a mild buzzing and ringing sound that can be ignored to extremely loud persistent and uncomfortable sounds that become debilitating to the afflicted, oftentimes resulting in a severe reduction of their functional capability. It is estimated that more than 2.7 million people have tinnitus that would be categorized as profound in severity and interferes with their ability to function normally.

Currently there are no broad-based gold standard treatments for tinnitus. It has been suspected that neural stimulation may be effective in the treatment and suppression of tinnitus symptoms. For example, cochlear implant users have reported symptomatic relief from their tinnitus, which may be due to the electrical stimulation delivered by their implant. There are a number of encouraging studies that demonstrate the benefits of electrical stimulation to treat tinnitus using cochlear implants; however, there have never been any specific studies using a stimulator with stimulation pulse parameters specifically designed for the treatment of tinnitus. Furthermore, the inventors are not aware of any dedicated systems for the effective treatment of tinnitus. Existing electrical systems for the treatment of tinnitus include modified cochlear implants, and electrical systems that stimulate either brain regions, or regions of the ear that are not in contact with the inner ear fluids (e.g., perilymph).

For example, US 2005/0080473 to Gibson et al. describes a cochlear implant that may be adapted for use to mask or treat tinnitus. However, the Gibson device is intended only for only extraluminar insertion. Furthermore this device does not allow for modification of the stimulation which may be necessary to avoid refraction and dose control. Other cochlear implants that have been modified to treat tinnitus typically include additional microphones or other sound transducing elements which are may be counter-indicated for treating tinnitus.

US 2007/0021804 to Maltan et al. describes a microstimuator to treat tinnitus, however, like the Gibson et al. reference, this devices is implanted only in front of the round window of the cochlea, and does not enter the perilymph. In addition, the electrical stimulation is not sufficiently adjustable to avoid refraction and dose control. Similarly, US 2007/0213787 (both to Kuzma et al.) also describes a system including a middle-ear electrode that may be used to treat tinnitus.

Unlike the devices described above, an effective stimulator designed specifically to treat tinnitus, based on our current understanding of this disease, would need to differ substantially from a cochlear implant, and should address problems that are specific to the treatment of tinnitus. In particular, such a system should allow control and adaptability of the treatment stimulation. In particular, the system should include a controller that allows the applied treatment signal to be adjusted in frequency, duration, intensity, on-time/off-time, and other stimulation parameters. The controller should be adjustable either manually (by a user or a physician) or automatically. The system also preferably allows for direct stimulation of the fluids of the inner ear (e.g., perilymph). In addition, the system should not include a sound transducer (such as a microphone or the like) as would be present in a typical cochlear implant.

For example, a system for treating tinnitus should address stimulation effectiveness. There are a wide range of conditions leading to tinnitus and it is unlikely that a narrow set of fundamental stimulation parameters will work on all subjects. It is an objective of the proposed system and devices to provide a flexible system and stimulation protocol that may be easily modified to provide the best results for each individual.

As mentioned above, a system for treating tinnitus may also prevent or correct therapeutic refraction. Experience with the use of electrical stimulation has shown that treatment can become refractive; it can lose therapeutic effectiveness with time. The systems and devices describe herein may allow adjustments to stimulation patterns, stimulation location and/or stimulation rest periods that may be helpful in reducing or eliminating these problems. The proposed system may incorporate methods to automatically alter electrical stimulation and field parameters to reduce or eliminate therapeutic refraction conditions.

In addition, the proposed device and systems described herein may allow dose control. A normal process for the treatment of tinnitus patients requires them to have frequent clinic visits allowing the clinical staff to do examinations and adjust their therapy. This is a costly and inefficient process. The proposed system described herein may incorporate novel programming methods that provide increase or decrease in the dose stimulation parameters over long time periods. This will result in fewer clinic visits and improved treatment outcomes.

Also as mentioned, the proposed system may also address some of the problems described above by allowing patient control of parameters: Clinical research reports have indicated that increased treatment effectiveness occurs when patients have control over the stimulation therapy they receive. It is an objective of this system to provide a remote control for patient use that allows them to adjust some stimulation parameters within safe boundaries, which are established and set at the treating clinic. These systems and devices may also include product safety features. For example, the proposed system may incorporate controls or limits to ensure the system is safe and cannot be misused by patients and others in the field.

Many of those with profound tinnitus have intact residual hearing with partial to full hearing loss. Those with profound hearing losses will most likely receive a cochlear implant and this device can be used to treat their tinnitus. The remainder will need a device that is very atraumatic and safe posing minimal risk to intact residual hearing.

The systems described herein may include one or more electrodes configured to provide a dedicated method for delivering electrical stimulation signals to the inner ear fluids to treat tinnitus while minimizing insertion trauma. This electrode is also intended to accommodate easy and straightforward surgical insertion and fixation in the hands of neurotologists and otolaryngologists with broad ranges of surgical experience.

Thus, described below are devices, system and methods that may address some of the problems and features mentioned above.

### SUMMARY OF THE INVENTION

The present invention relates to systems, devices and methods for stimulation of the cochlea that are sufficient to mimic or replace the spontaneous background neural activity of the cochlea thereby reducing or eliminating tinnitus. The inventors have hypothesized that the restoration of an approximately normal level of spontaneous background neural activity (e.g., neural activity that is not correlated to external sounds) in the cochlea may prevent or alleviate tinnitus. In some variations the systems and devices described herein may sense the level of spontaneous neural activity in the cochlea of a subject suffering from tinnitus and supplement it to approximate or mimic a more normal level of spontaneous (“background”) spontaneous activity. Thus, the systems and device may be configured to sense the spontaneous cochlear neural activity (including receiving electrical channel or channels). In some variations this means that the system is configured to apply a pattern of current pulses that will evoke a distribution (e.g., pattern) of neural activity in the cochlea that is similar to the pattern of normal spontaneous activity in the cochlea. In some variations the system is configured to apply a pulse train of current frequencies that will evoke an average frequency of neural activity that has a distribution similar to normal spontaneous activity in the cochlea. A normal pattern of spontaneous activity in the cochlea may be determined from the individual (e.g., during periods when tinnitus is suppressed or eliminated) or from recordings taken from similar populations of tinnitus-free individuals (e.g., as an average, composite, or the like). Thus, the system may use a target ‘normal’ level of spontaneous (baseline) activity. More than one target level of spontaneous activity may be used. For example, if the spontaneous level is context-dependent, the system may be adapted to modify the pulse train of applied current based on various context-specific target levels. Furthermore, the applied current pulse train may be adjusted (to adjust the duration of the pulses, the inter-pulse interval, the burst duration, the burst on-time, the burst off-time, etc.). In some variations the subject may adjust the applied current pulse train (e.g., within safety parameters) to allow the subject the subject to directly respond (provide feedback) on the perception of tinnitus.

For example, described herein are systems for treating tinnitus by electrically stimulating the cochlea to supplement the baseline spontaneous neural activity of a subject's cochlea. These systems may include: an implantable lead configured for insertion through the round window of the cochlea so that one or more electrical contacts at the distal end of the lead is within the cochlear scala tympani to a depth of 1 mm or less; a signal generator configured to deliver a train of current pulses; and a controller coupled to the signal generator and configured to modify the train of current pulses from the signal generator so that the current applied by the implanted lead triggers a pattern of cochlear stimulation that is similar to the baseline spontaneous neural activity of a normal cochlea.

The lead may comprises a sharp distal end configured to penetrate the round window of the cochlea. In some variations, the lead includes a stop located proximally about 1 mm or less from the distal end of the lead. The implantable lead may comprises a plurality of electrical channels and the system further comprises a multiplexer coupled to the plurality of channels (e.g., two channels, three channels, four channels, etc.).

As mentioned, the controller may be adjustable to adjust the pattern of current pulses. In some variations, the controller is adjustable by a subject wearing the implantable lead.

The signal generator may be part of an implantable therapeutic stimulator configured to couple with the implanted lead. Similarly, the controller may be part of a wearable head-level processor. In some variations the controller is also part of the implantable therapeutic stimulator.

The signal generator may form part of a stimulator including a pulse shape modulator, a burst mode modulator and a dose control modulator, further wherein the controller is configured to control the pulse shape modulator, burst mode modulator and dose control modulator.

In general, the systems described herein are distinguishable from existing cochlear implants in a number of ways. For example, in general, the system for treating tinnitus described herein do not include a microphone (e.g., a sound transducer or the like). In particular, the systems do not transduce sounds from the external environment (speech, etc.) and relay them into the signal provided to the cochlea. However, external sounds (e.g., noise level, etc.) may be used to modify the applied current train (e.g., if spontaneous neural activity in the cochlea is related to noise level, for example).

Also describe herein are systems for treating tinnitus by electrically stimulating the cochlea to supplement the baseline spontaneous neural activity of a subject's cochlea that include: an implantable lead having one or more electrodes, the lead configured for insertion of the one or more electrodes through the round window into the cochlear scala tympani; a stimulator configure to apply current to the implantable lead, the stimulator comprising a pulse generator configured to emit a train of current pulses, a pulse shape modulator configured to modulate the shape of the current pulses emitted by the pulse generator, a burst mode modulator configured to modulate the emitted train of current pulses to an adjustable burst on-time and burst off-time, and a dose control modulator configured to modulate the emitted train of current pulses to an adjustable dose level; and a controller configured to control the dose control modulator, burst modulator and pulse shape modulator to emit a pattern of current pulses from the implantable lead that trigger neural activity in a subject's cochlea having a pattern similar to a baseline spontaneous neural activity pattern. Any of the features described above may be included in these systems.

Also described herein are methods of treating tinnitus. In particular, described herein are methods of treating tinnitus by electrically stimulating the cochlea to mimic the baseline spontaneous neural activity of a subject's cochlea, the method comprising: inserting an electrical lead within the cochlea; and applying current pulses within the cochlea from the electrical lead to trigger neural activity that mimics baseline spontaneous neural activity of the subject's cochlea.

The step of inserting may comprise inserting the electrical lead through the round window and into the cochlear scala tympani. For example, the method may include the steps of implanting the lead so that one or more electrical contacts on the lead extend into the cochlear scala tympani 1 mm or less from the round window of the cochlea.

The step of applying current pulses may comprise applying a train of current pulses between about 3 and 5 kHz, which may be a frequency range within the spontaneous (baseline) level. The method may also include the step of sensing the baseline spontaneous neural activity of the subject's cochlea.

The step of applying current pulses may comprise sensing the spontaneous neural activity of the subject's cochlea and comparing the neural activity to a predetermined target level of baseline spontaneous neural activity. In some variations, the step of applying current pulses comprises allowing the user to adjust the dosage of the applied current pulses.

Also described herein are methods of treating tinnitus by electrically stimulating the cochlea to supplement the baseline spontaneous neural activity of a subject's cochlea comprising: inserting an electrical lead within the cochlea; sensing the spontaneous neural activity of the subject's cochlea; and applying current pulses within the cochlea from the electrical lead to supplement the spontaneous neural activity of the subject's cochlea and reduce the tinnitus.

### BRIEF DESCRIPTION OF THE DRAWINGS

FIG. 1 illustrates one variation of a system for treating tinnitus, as described herein.

FIG. 2 is another variation of a system for treating tinnitus.

FIG. 3 is another variation of a system for treating tinnitus including programming tools.

FIG. 4 illustrates an operating room diagnostic tool for use with the systems described herein.

FIG. 5 illustrates a system and component test module.

FIG. 6 is another variation of a system and component test module.

FIG. 7A illustrate one variation of a single-channel tinnitus electrode and lead as described herein. FIG. 7B is a front view of the distal tip region of the lead of FIG. 7A.

FIG. 8 is another variation of a tinnitus electrode and lead, having two channels.

FIG. 9 is another variation of a tinnitus electrode and lead.

FIG. 10 is a block diagram schematically illustrating a controller for a system as described herein.

FIGS. 11 and 12 illustrate exemplary pulse trains from a pulse generator portion of the systems described herein.

FIG. 13 illustrates an exemplary pulse train that has been shape modulated.

FIG. 14 illustrates one variation of a stream of pulses that is burst modulated (e.g., stimulation pulses).

FIG. 15 illustrates one variation of a stream of pulses that has been does modulated.

### DETAILED DESCRIPTION OF THE INVENTION

The inventors have hypothesized that tinnitus may be caused (at least in part) by a decrease in the spontaneous neural activity that is normally present in the cochlea. This normal neural activity, which may be referred to as “baseline” or “normal spontaneous” activity is interpreted by the brain as the perception of silence, and a loss of this spontaneous activity may result in the brain attempting to compensate by increasing the effective amplification in an effort to compensate for the loss. As a result of this attempted amplification, a ‘ringing,’ buzzing, or other illusory noise is experienced, commonly referred to as tinnitus.

Described herein are systems, devices and methods for treating tinnitus. In particular, described herein are systems for treating tinnitus by providing controlled electrical stimulation to the perilymph of the cochlea in order to reestablish an apparently normal spontaneous level of neural activity from the cochlea. As described herein, this target ‘baseline’ level of activity may be referred to as uncorrelated neural activity, because it is not correlated with the presences of a noise. Thus, it is uncorrelated to a particular sound.

These systems described herein may include a head-level processor (which may be worn externally or implanted), an implanted therapeutic stimulator (ITS) and a stimulation electrode configured to deliver electrical stimulation signals to the inner ear fluids (e.g., perilymph). The system may also include a power supply, or it may be supplied by an external power source (e.g., via induction). In variations in which the head-level processor is external (e.g., worn over or behind an ear), the system may also include a headpiece connected to the implanted therapeutic stimulator.

In general, the devices and systems described herein may include a controller (or processor with a controller) for applying electrical signals that trigger cochlear electrical activity that mimics a normal baseline spontaneous level of neural activity perceived as silence. As used herein the level or pattern of neural activity that is “normal” may be determined based on an average (e.g., from a particular patient population) or it may be based on measurements taken from one or more subjects.

The controller typically controls the applied electrical energy. The energy may be applied as one or a train of pulses. The pulse train may be controlled so that the pattern of pulses, the rate of the pulses and the intensity (e.g., level of modulation) are all regulated to treat tinnitus. In some variations, the applied pulses are triggered in an irregular pattern (which may be random or preselected). In some variations, the pattern of applied pulses may be modified by a user or by a physician. Thus, the system may include one or more user or physician inputs, or may include an input line for receiving instructions (from a user or physician) to modify the applied electrical pulses to the subject. The systems described herein may thereby provide a flexible stimulation protocol that is easily modified in the clinic to provide the best results for each individual.

The controller may be included as part of the head-level processor or as part of the headpiece and cable, or its functions may be distributed between the two. In some variations, the head-level processor includes a controller, a program module (for receiving and/or processing instructions for applying stimulation), and a signal generator that is controlled by the controller. The program module may be part of the controller, and the controller typically receives instructions from the program module. Inputs from users/physicians may be sent to the program module. Thus, in some variations the head-level processor includes an communications module (e.g., including telemetry or other signal input).

The controller is also typically configured to allow adjustments to the stimulus applied (e.g., to the stimulation patterns applied by the signal generator). The system describe herein may be configured to automatically alter electrical stimulation and field parameters based on input from a user or from one or more sensors. For example, the system may be configured to detect baseline electrical activity within the cochlea (e.g., the spontaneous neural activity that is present in the cochlea). Based on the detected endogenous baseline spontaneous neural activity in the cochlea, the system may provide additional electrical stimulation so that the non-correlated neural activity (e.g., activity that is not correlated with hearing an audible sound) is approximately that of a predetermined level, such an average “normal” level.

Thus, the system described herein may replace lost spontaneous or baseline activity. In some variations the stimulation applied by the system is applied without sensing existing or ongoing baseline activity.

In some variations of the system described herein, the system allows for the increase or decrease in the dose stimulation parameters over long time periods. Thus, the controller or processor may include instructions for adjusting the dose (applied current) over time, either in response to input (including user input) or based solely on timing.

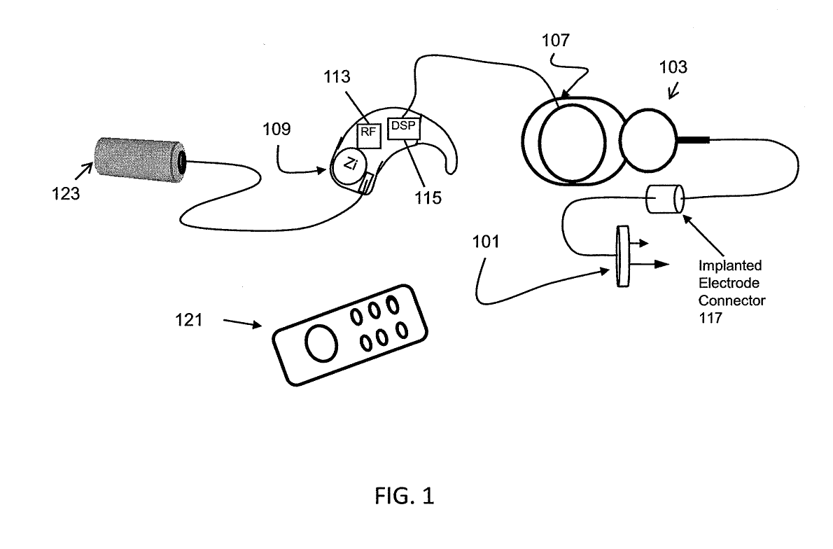

Various embodiments of the system described herein are illustrated below. For example, FIG. 1 shows one example of a system that may be used to treat tinnitus. In particular, a system may include a tinnitus electrode (e.g., an electrode adapted for treatment of tinnitus, and for insertion to apply electrical energy within a fluid of the ear, such as the perilymph), and a stimulator adapted for applying electrical energy to the ear through the electrode. The stimulator may be particularly adapted to provide multiple levels of modulation of the applied electrical output, in a range that has been observed to be effective for the treatment of tinnitus.

The systems described herein may be configured for temporary (acute) use and long-term (chronically implanted) systems. The system illustrated in FIG. 1 is configured for long-term (implanted) use, and thus may be referred to as a permanent implantable stimulator system.

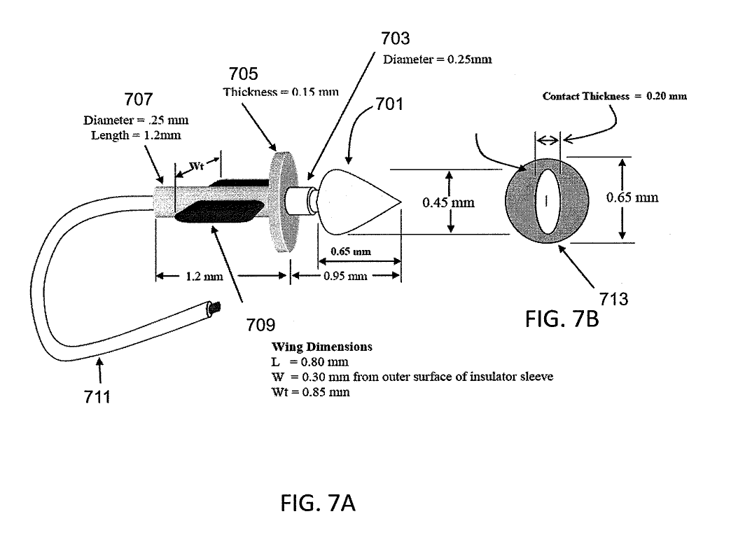

In this variation, a permanent implant (implanted electrode 101) is attached to the round window of the cochlea so that the electrodes project into the perilymph. This electrode may provide stimulation to treat a broad range of tinnitus conditions and symptoms. The electrode (lead) 101 may include a plurality of contacts, or it may include a single contact (see FIGS. 7-9 below for examples of leads that may be used). The lead 101 in FIG. 1 is permanently implanted, and may be connected directly to an implanted therapeutic stimulator (ITS 103), or it may be connected via a connector (implanted electrode connector 105) as shown in FIG. 1. The implanted therapeutic stimulator 103 in this example delivers the automated stimulation control required to vary the dose, modify stimulation patterns and overcome refractive therapeutic reactions. The system of FIG. 1 also includes an implantable pulse generator, and a lead/contact set enclosed in a hermetic biocompatible housing (headpiece 107). In some variations the headpiece 107 and the ITS 103 are integrally connected or formed as a single device performing both (or some of both) functions.

The system may also include a head-level processor 109. In FIG. 1, the head-level processor includes a housing that encloses an external head mounted power source 111 (shown a as a battery), a system controller, an RF driver 113, a programming interface (digital signal processor 115). The programming interface and the controller may be combined, or the two may be separate elements. In some variations, the head-level processor may include or be connectable to a cable (or it may be wirelessly connectable) to the headpiece to provide energy and/or data to the implanted device. In some variations a remote patient controller 121 may also be included. A supplemental or auxiliary power supply 123 may be included as well. The auxiliary external battery pack may be particularly helpful in situations requiring very high power or extended operating time. Programming cables (e.g., for connection to programming modules, as described below), manuals and packaging materials may also be included.

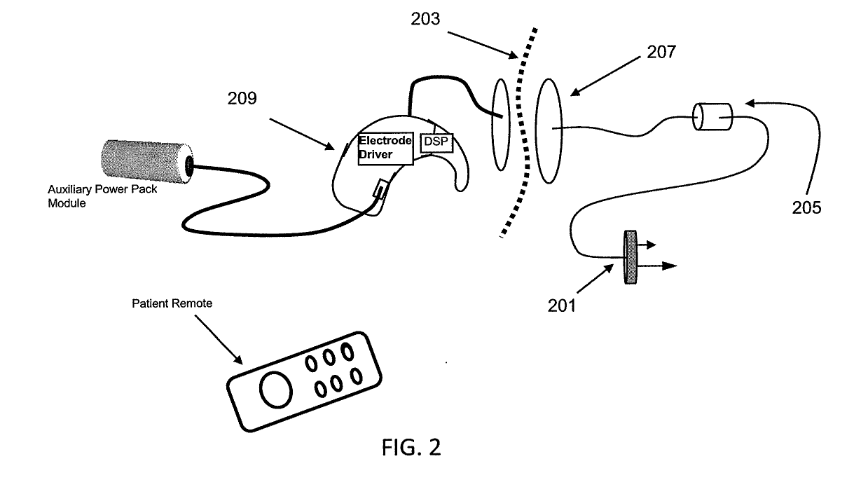

FIG. 2 illustrates another variation of a tinnitus treatment system. In this example, the system is configured as a temporary stimulator that may be used as a screening tool to determine if a subjects would benefit from the stimulation therapy described herein. This variation may not require the extended life of a permanent implant, although some components may overlap or be compatible with components for a longer-term implantable system such as the one described above for FIG. 1. Additionally, the electrode in this example may be removable if it is determined that the subject cannot benefit from the permanent implant.

In the example shown in FIG. 2, stimulation energy is provided by the external coil drivers coupled through the skin 203 to an internal broadband coil 207 that is connected to the electrode 201 through an implantable connector 205. The system may be otherwise similar to the variation described above. If the subject using the system of FIG. 2 has a beneficial response to therapeutic stimulation, the electrode 201 may remain in place while the internal coil 207 and lead is removed. A permanent implant (e.g., the ITS 103 of FIG. 1) can then be connected to the existing lead and electrode during implantation of the permanent device.

The variation shown in FIG. 2 may also includes a controller, a pulse generator, program module, and the like, in addition to an external coil/antenna driver(s) located in an external head mounted package, such as a head-level processor 209. A remote patient controller 221 may also be included, as well as a programming module (not shown), cables, manuals and the like. The stimulation capabilities of a temporary implant may not be as broad as the permanent implant, but may be sufficient to assess the subject's response to therapeutic stimulation. However, any of the components of the “temporary” system described herein (e.g., the external inductive power supply) may be used as part of a long-term or permanent system, and vice-versa.

In any of the systems described herein the systems is configured to generate stimulation patterns to be applied to the electrodes to alleviate tinnitus, preferably by generating a normal baseline level of spontaneous stimulation in the cochlea.

In some variations, this may be achieved by providing a high-rate repetition frequency (e.g., PRF>16,000 PPS per channel), narrow pulse-width stimulation. This type of stimulation is distinct from the stimulation protocols applied in cochlear implants designed to restore hearing.

The primary component of the many of the systems described herein includes a programmable pulse generator that is connected to a lead/electrode contact that can apply electrical pulses to the cochlea to elicit a neural response. The programmable pulse generator may be part of the controller, as mentioned above, and may be enclosed in the head-level processor (external) component or it may be part of the implantable therapeutic stimulator component. For example, this system may include an internal computer and storage capability to allow clinicians to program stimulation parameters and set the limits of stimulation based upon the comfort tolerance of the patient. The system may restrict stimulation to safe charge density limits, so that the user or device fitting/programming will not be able to exceed these limits. Portions of the system (e.g., the ITS) may be housed in an implantable hermetic package with an internal lead.

In some variations the lead is an intracochlear ‘thumbtack’ electrode that is configured to penetrate the round window membrane of the cochlea for insertion into the cochlear scala tympani for a maximum depth of 1 mm. This intracochlear electrode typically enhances the electrical coupling to the internal cochlear structures, potentially increasing the effectiveness of the stimulation and reducing the energy needed to achieve tinnitus suppression.

The system may also include components that allow clinicians to test and program the devices. These include tools and software designed to aid surgeons in the proper placement and fixation of the device and to assess device function with the ability to test leads and electrodes just before and after implantation.

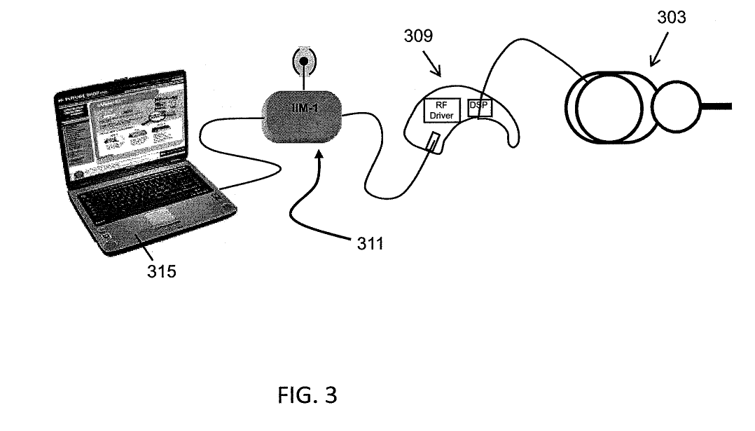

For example, FIG. 3 illustrates one variation of a system including testing and programming modules for the tinnitus treatment devices and systems. In FIG. 3, the system shown includes a programming interface module comprising an interface/isolation module (IIM) 311. The IIM provides a computer connection interface to the head level processor (HLP) 309. The module also isolates the signal to prevent shock and may provide power to the ITS. The IIM may be wired to connect or may wirelessly connect to the HLP and/or a computer 315. The computer 315 may be used to fit and program the device components of the system such as the HLP 309 and the ITS 303 to a subject in need of the device.

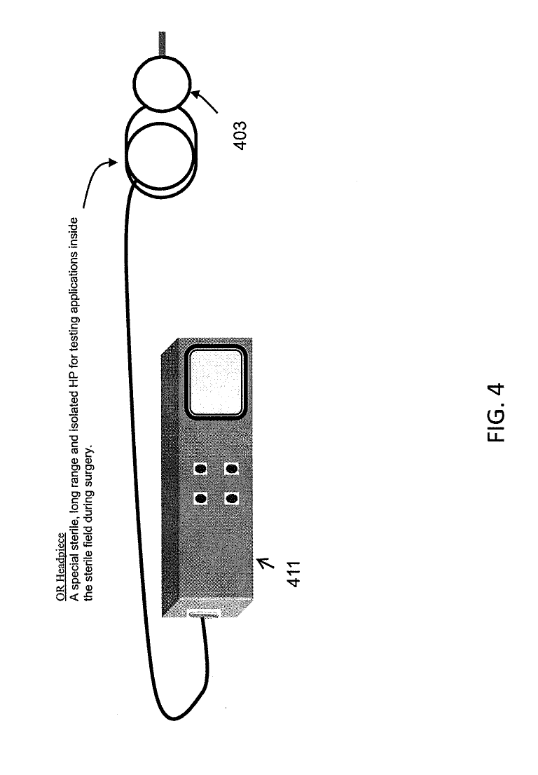

FIG. 4 shows another variation of a system including diagnostic components that may be used to test or configure device components of the tinnitus treatment system. For example, in FIG. 4, the device includes an operating room diagnostic tool (ORT) 411. The ORT may provide a diagnostic means for testing the ITS 403 prior to removal from sterile packaging, for example, or prior to implantation, after the electrode is inserted. This may reduce or eliminate the possibility of implanting a defective device. The ORT may test the device to determine the basic implant function, check communication with the various system components, test the electrode impedance of the inserted electrode, and the like. Other system diagnostics may also be run by the ORT, which may provide output (e.g., on screen or monitor) of the status. In some variations the ORT may allow the measurement, recording and/or display of ECAP signals. Thus, the ORT may allow observation of the baseline background neural stimulation in the cochlea, and may also determine the effectiveness of the applied electrical stimulation. The ORT may therefore be used to calibrate the system.

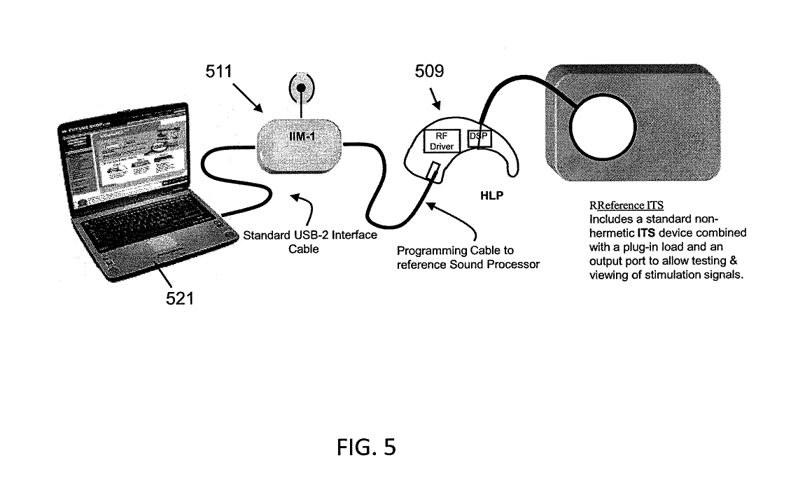

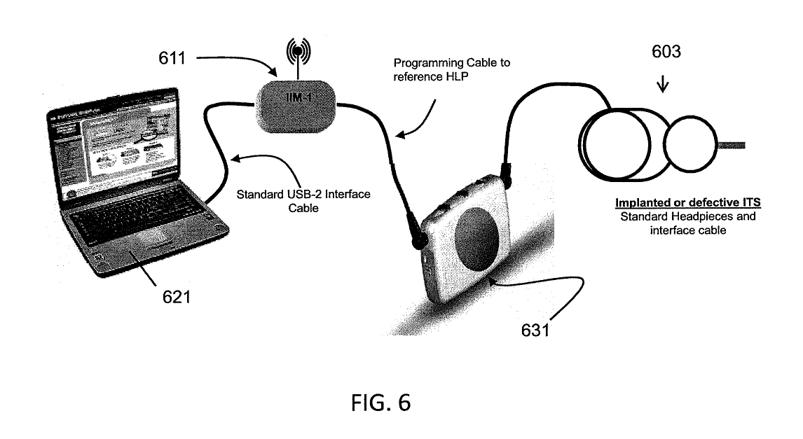

Other components of the system may also be tested by one or more devices. For example, FIG. 5 illustrates a system for testing an HLP 509. In FIG. 5, the HLP 509 is shown connected to an interface/isolation module (IIM) 511, which is connected to a computer 521. The HLP 509 is in turn connected to a reference ITS that provide a plug-in load and an output port to allow testing and viewing of sample stimulation signals. FIG. 6 show a similar arrangement for testing an ITS 603, using a reference head level processor (reference HLP) 631. The reference HLP may provide power to the ITS and may otherwise simulate the behavior of an HLP to allow testing of the ITS and other system components. The testing systems shown in FIGS. 5 and 6 allow the implant and external system components to be tested to diagnose problems even before the device are implanted, by providing reference components (e.g., reference ITS and reference HLP) to the systems shown.

Any of these systems may include hardware, software or firmware for programming, testing and operating these device components of the tinnitus treatment systems. For example, the system may be configured to run an embedded operating system (EOS) containing the stimulation functions, therapeutic options, diagnostic modes, and other tools and control codes for operating the system. In addition to the operating system, a clinical fitting and diagnostic system (CFDS) may also be used, which provides fitting software. For example, the system or components of the system may be connected to a computer to run the EOS (e.g., to communicate/program the various components) and/or the CFDS. This may allow the system to receive patient programs, set patient stimulation operating limits, activate the processor and other components of the system, and/or diagnose system problems. Additional software tools may be provided to access or control other system features, including the controller (e.g., setting the timing, duration, pattern, etc. for stimulation).

### Exemplary Hardware and System Configurations

In operation, the systems described herein may be configured in one of several ways to treat tinnitus, diagnose problems with the system, and to assess patient response to the system. For example, a system such as the one shown in FIG. 1 or 2 may be configured as illustrated below.

In one example, the lead is a multi-channel lead, such as a four channel lead that drives four electrode contacts. The energy is applied as current, and can be driven as either four-channel monopolar or two-channel bipolar stimulation. The current can be drive at a 10 bit resolution of up to two milliamps (the maximum current range may be selectable in different ranges). The resolution of the amplitude step may be approximately 1 microamp. In this example, the voltage supplying the system is a 12 volt power supply, and the maximum pulse voltage is approximately 11.5 volts. The pulse width of the stimulation pulse is between about 1 microsecond and 300 microseconds. The PRF (Pulse Repetition Frequency) is about 16,000 pulses per second, where the max stimulation rate for each channel is the PRF divided by the number of active channels. The parameters for the pulse shape may be selected from pre-set shapes including rectangular, trapezoidal or triangular. The minimum rise/fall time for the pulses is approximately 500 nsec. The modulation functions and types may include multiple AM, FM, PPM, FSK, PSK, preset modulation functions. Custom modulation sequences are also possible; the system has the ability to accept several custom modulation functions plus several programmable custom functions with arbitrary variable modulation patterns. For example, these functions can be provided through the operating software loaded into the system.

In this example, the modulation rates are typically between 0.01 to 1000 cycles per second. An exemplary short gated sequence (SGS) includes a burst mode of modulated groups based on the ratio of times needed for one carrier cycle. For example, on-time may be 1 to an infinite number of cycles (e.g., on full time). Off-time may be zero to 10,000 cycles. An exemplary long gated sequence (LGS) includes gated groups of burst mode stimulation based on the group cycle time ratio. In this case, the on-time lower limit is one group cycle and the off-time is up to 10,000 group cycles. The stimulation may also be variably controlled. Thus, the output may be variably time controlled. For example, the variable time control for an LGS allows the off-time between stimulation cycles to be varied over time. This may provide the ability to effectively reduce the dose as a function of time. For example, VTC (variable time control) may be between about 1.0 and about 10, where this dimension of VTC is complete cycles defined by the SGS. The signals may also be variably amplitude controlled. For example, a variable amplitude control for the LGS (LGS-VAC) allows the system to vary the amplitude of the stimulation based on the number of stimulation sequences delivered to the patient. This may allow the use of a pre-programmed function that decreases does with time, increases dose with time, or turns off the dose completely after a specified total does (the total number of pulses, pulse cycles or total charge) is delivered. For example, the amplitude may be varied between about 0.5 to about 0.999 (STM(t)=VAC*(t)*STM(−t).

In this example, the contact impedance of the internal electrode contact is approximately 3000 ohms for the penetrating electrode. In variations of the system described herein that use a non-penetrating electrode (e.g., a temporary electrode outside of the round window), the contact impedance of such an extra-cochlear electrode may be greater than 10000 ohms.

In any of the systems described herein, a multiple-channel architecture may provide a back-up option in the event a channel or an electrode failure occurs or an open or shorted lead is discovered. Additionally, it may provide the ability to combine two or more channels differentially to focus and localize stimulation energy. The system may also deliver low-level stimulation on one channel combined with higher-level modulated stimulation on another channel. The architecture also makes it possible to have an ECAP measurement added in the feature. This may requires at least two independent channels that are spatially separated. Examples of this are provided below.

The systems described herein may include sufficient processing power to allow multiple stimulation programs to be available to a user or clinician. For example, up to 10 pre-set stimulation programs may be available for selection by a user or uploaded/downloaded for use onto the system. In operation, any of the parameters for stimulation may be modified or defaulted to a pre-set value or range of values. For example, the maximum/minimum amplitude of the stimulation, an amplitude attenuation factor, a maximum/minimum stimulation rate (PRF range), the SGS on/off ratio, the LGS on/off ratio, and the LGS off time sequence may all be set or chosen from a predetermined menu of values.

As mentioned, the stimulator components of the system may comprise a portion of the external head level processor (HLP) component, or it may be part of the internal/implantable therapeutic stimulator (ITS), or may be distributed between the two. The stimulator may comprise a controller as described above, as well as a signal generator controlled by the controller and additional signal conditioning elements controlled by the controller.

A stimulator typically provides multiple levels of pulse generation and modulation to output a pattern, rate and level of modulation in a range that is consistent with the effective treatment of tinnitus. These stimulators may also include inputs and control over various stimulation parameters providing modulation of variables effective for the prevention of refraction during treatment, to allow controlled treatment dosages, and allow patient control of treatment within controlled bounds, and to allow product safety parameters.

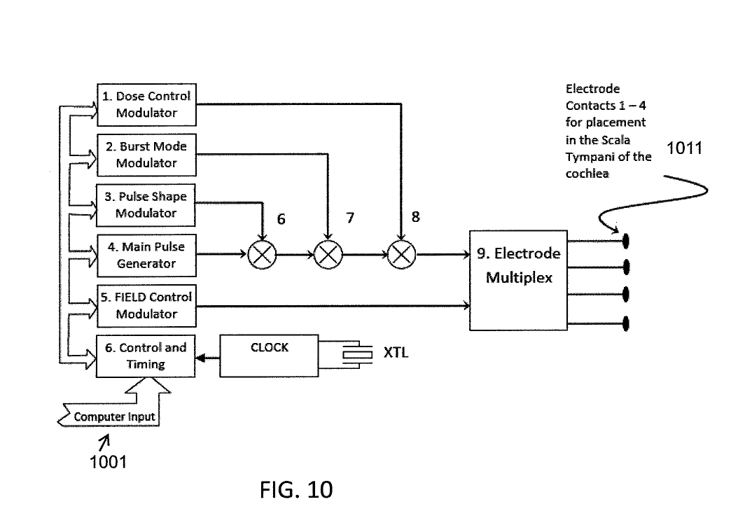

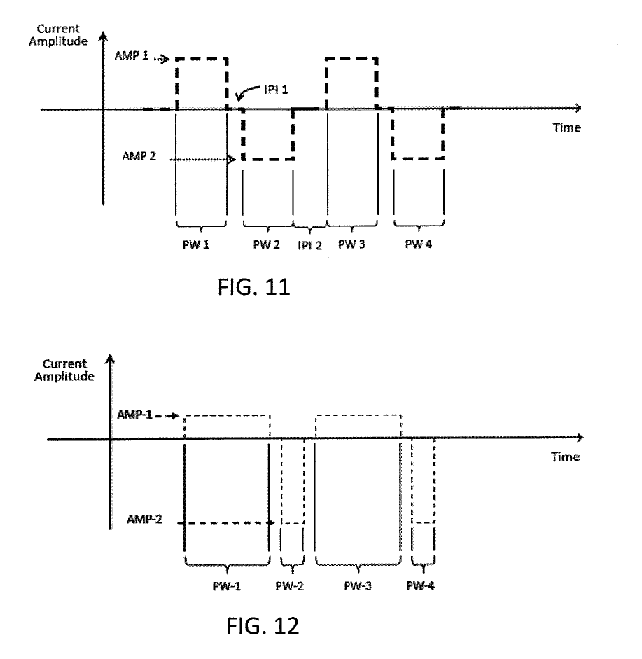

Turning now to FIG. 10, an exemplary stimulator may include component elements that modulate the pulses emitted by a pulse generator. The controller may control (e.g., trigger) the pulse generator and the component elements. FIG. 10 shows one variation of a basic system block diagram and the names of the key control blocks. The controller (computer input 1001) may trigger, control and coordinate the activity of these control blocks. FIGS. 11-15 illustrate the various effects of these different elements or components.

For example, the main pulse generator 4 in FIG. 10 may generate a continuous pulse sequence made up of an equal number of positive and negative phase current pulses (biphasic current) separated be an inter-pulse interval of zero current. Biphasic current may be used to avoid charge build-up, however monophasic current may also be applied in some variations.

In this example, the signal includes a train of pulses (square pulses) separated by inter-pulse intervals. The different pulses of the train are each labeled as PW-n (for pulse within number n) and each pulse is separated by an inter-pulse interval (IPI) labeled IPI-n. For example, PW-1 is the first pulse of pulse sequence. This pulse may be, for example, between about 5 and about 1000 microseconds in duration. IPI-1 is the first inter-pulse-interval, and may be between about 1 and about 100 microseconds in duration. The first pulse has a (positive) amplitude of AMP-1, and the second pulse (PW-2) has a similar shape and duration (e.g., between about 5 and 1000 microseconds) and an amplitude (negative) of AMP-2. The second inter-pulse interval separates the second pulse from the next sets of pulses (PW-3, IPI-3, PW-4, IPI-4, etc.). The maximum frequency (F.) from this exemplary main pulse generator is approximately equal to the 1/(PW−1+IPI−1+PW−2). By this definition the maximum frequency could exceed 90 MHz, but, for practical reasons, it may be limited to lower values.

The pulse sequence may be constrained to include an equal number of positive and negative phases. The pulse-width times and pulse amplitudes can be different for positive and negative phases; however, the amplitude-time product may be the same for both phases to balance the charge delivered to the tissue, as illustrated in FIG. 12. In this example:

(AMP−1* PW−1)=(AMP−2*PW−2),

and

PW−3=PW−1 and PW−4=PW−2.

This variation, in which the amplitude of the negative pulse is greater than the amplitude of the positive pulse.

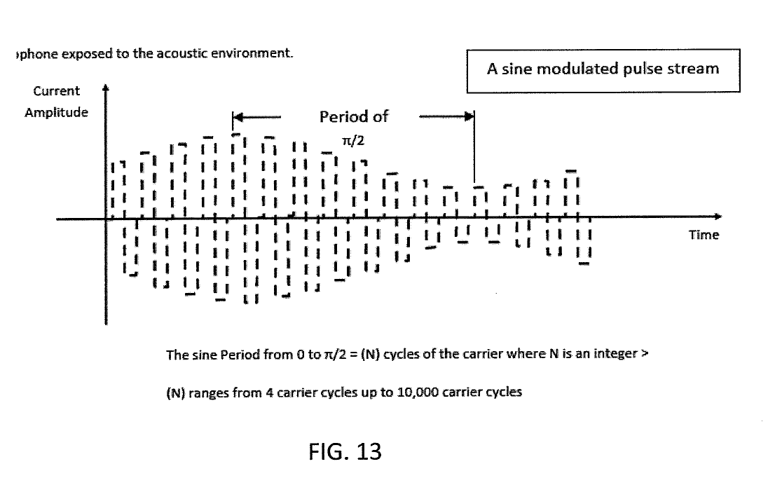

Returning now to FIG. 10, the stimulator may include a pulse shape modulator 3 that modulates the amplitude, pulse position, phase or frequency of the fundamental pulse sequence. The frequency of this modulation is generally much lower than the frequency of the main pulse sequence (the carrier sequence). This modulator can apply standard modulation functions ranging from sine and cosine to functions like SIN(X)/(X) or other complex window functions. Additionally, the controller/stimulator may be configured so that a programmer may develop and download to the system an arbitrary waveform function that can be used to modulate the continuous pulse sequence. FIG. 13 shows an example of a sine-modulated pulse stream. Although the systems described herein typically do not include a sound transducer, in some variations a modulation signal could be derived from a band limited acoustic signal received and amplified from an external microphone exposed to the acoustic environment.

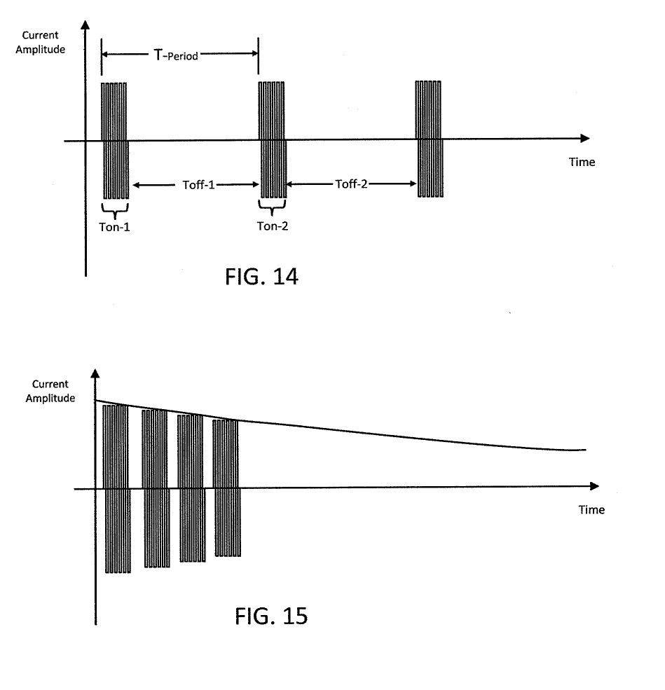

The system shown in FIG. 10 also includes a burst mode modulator or short-gated-sequence (SGS) that can gate the modulated pulse sequences generated by the pulse generator (4) and modulator (3) described above. The burst generator may provide on- and off-times equal to an integer number of complete cycles derived from either the pulse generator or the pulse shape modulator. The burst generator can determine its on/off timing information from a cycle count derived from the pulse shape modulator if it is active; otherwise, it can default to the cycle count derived from the main pulse generator. In some variations, the duration of the on times and off times can be varied over time algorithmically so that they change with time or cycle count. This is illustrated in FIG. 14, showing a set of three bursts. Each burst has an on time (e.g., Tₒₙ−1, Tₒₙ−2, etc.) and an inter-burst duration (T_{off}time of T_{off}−1, T_{off}−2, etc.). In general the duty cycle applied (duty cycle is the time on/the total period, or time/(time on plus time off)) may vary between 100% (always on) and about 0.1%. In some variations the time off and time on may be varied. For example, the time on may vary between 1 and 10,000 cycles, and the time off may vary between 0 cycles and 1,000,000 cycles.

As mentioned, the applied sequence of pulses may be charge-balanced, so that the stimulation is charged-balanced; having equal positive and negative phases during the burst on-times. Burst mode stimulation may provide time sequences with durations of several minutes up to a few days.

The dose control modulator (1) or long-gated-sequence (LGS) portion of the stimulator/controller (shown in FIG. 10) may provide a means to vary stimulation over long periods of time to approximate a change in dose that might be made in the clinic. The time scale of this element typically ranges from a few hours to several weeks in duration. The dose control can modify several parameters over time, including the amplitude and pulse width of the main pulse generator, the modulation functions used, the modulation frequency and amplitude and the burst mode sequence. An embedded system clock (1003 in FIG. 10) may provide the timing for the dose control modulator, and the timing for the pulse generator/controller may be coordinated by the control and timing element (6). The pulse sequences may be stored in system memory or may be algorithmically derived based on clock pulse number count or an absolute time count. The dose control modulator may provide a means to prevent therapeutic refraction along with a means to determine if the subject can tolerate a reduction in stimulation over time while maintaining a beneficial reduction in tinnitus symptoms. The dose control modulator may be adjusted to effect the overall dose applied by the system, including decreasing and/or increasing the overall dose. The dose control modulator may have priority over all other functions of the stimulator. The system may include a memory or storage to log the dose, the time the dose was modified and the duration of dose delivery. This log can be retrieved and reviewed by the treating clinic, and may also be used by the different functions of the device. FIG>15 illustrates a trace showing the effect of one variation of a does control modulator that reduces the amplitude of the bursts of pulses over time; the time scale is on the order of weeks. The does control modulator (or any of the components described above) may be adjusted, e.g., by the controller, based on feedback measured from the cochlea, or from the patient, or both. For example, the controller may increase or decrease the applied stimulation (duration, inter-pulse interval, amplitude, Tₒₙ, Tₒₙ, combinations of these, etc.) based on the level of baseline (noncorrelated) stimulation compared to an indicator of normal spontaneous electrical activity in the cochlea, as mentioned above.

The system (and particularly the controller/stimulator) may also include a field control modulator (5) element and an electrode multiplex (9) which may provide a means to modify the shape of the electric field that stimulates the neurons based on the components described above. For example, the electrode multiplex may determine which electrodes on the lead (and potentially which nearby neurons) get stimulated at different times in the sequence. IN the variation shown in FIG. 10, the lead includes four electrodes 1011, each of which may be stimulated by the stimulator in a distribution pattern determined by the multiplexer. In some variations only a single electrode (or electrode pair) is included on the lead, and therefore a multiplexer is not necessary.

In some variations the system (controller/stimulator portion) also includes a field modulator (5) that can select which electrode configuration to stimulate, based on an amplitude weighting on each electrode contact. The sequencer may be synchronized to any signal generator block to provide field modulation times that varies from very short time durations to very long time durations depending on the needs of the subject.

As mentioned above, the system may also include a remote control. The remote control may interface with the controller/stimulator to allow the patient or clinician to modify the pulse parameters. This control may allow adjustments for the amplitude of the stimulation and for the frequency of stimulation. It may provide an option that allows the patient to override stimulation-shut-down initiated by the dose control modulator. This override can be limited in duration and the patient is required to contact their clinic for a programming change. The range and limits of all parameters may be set at the clinic to values that are determine to be safe and effective for the patient. This remote control may be configured so that it does not allow the patient to modify the pulse shape modulator or other parameters to exceed some max values.

In some variations, the system may also be configured to allow drug delivery. For example, the system may be configured to include a reservoir and pump controlled by the existing embedded processor and system control software. This may provide the ability to add a site-specific drug delivery system and to synchronize drug delivery and dose with stimulation patterns to enhance the effectiveness of this drug therapy.

### Lead and Electrodes

Also described herein are leads appropriate for use with the systems for treating tinnitus. In general, each lead includes one or more electrodes and these leads are particularly well suited for use in treating tinnitus. The leads are configured for insertion thorough the round window of a cochlea, so that the electrical contact surface or surfaces (in variations having multiple electrode contacts) may be placed in communication with the perilymph or other fluids of the ear. The lead may be applied using standard techniques (including cochlear implant techniques) and may be connected to stimulator for treating tinnitus, and particularly stimulators that are specifically designed to treat tinnitus.

An objective of the lead and electrode design(s) described herein is to provide a means to puncture the round window without the need for an incision, although an incision may be used if the surgeon desires. Another objective of this design is to minimize the potential for residual hearing loss by limiting the insertion depth and length of the internal electrode structures. Since the target treatment patients may have only mild to moderate hearing loss, the lead should preserve as much of this residual hearing as possible.

In general, these leads are stabilizing (or stability) leads for the round window of the cochlea that stabilize the electrode in the round window, yet allow relatively atraumatic introduction, reduced surgical time, and increased device safety. The stabilizing leads for the round window of the cochlea described herein allow insertion with the tip of the lead oriented vertically then rotated (e.g., approximately 30 degrees, 45 degrees, 70 degrees, 90 degrees, etc.) to secure the lead in place. Additionally, the electrode is configured to be removed by reversing this process and withdrawing the lead.

For example, the stabilizing leads described below and illustrated in FIGS. 7-9 may include one or more (e.g., opposing pairs of) grab wings that are provided for the surgeon to use standard surgical forceps of the type used for conventional middle and inner ear surgery without imposing the need for special or custom designed tools. The grab wings may provide a rotational orientation reference and are orientated at 90 degrees to the plane of the electrode to aid in placement and fixation.

Turning now to FIG. 7, a lead configured as a single-channel tinnitus lead has a sharp, flattened (in this example, flattened conical) distal tip configured to penetrate the round window of the cochlea. The dimensions given in FIG. 7 (and in any of the figures herein) are exemplary only, and are not intended to be limiting or necessary, unless the context or description clearly indicates otherwise. For example, the dimensions may be +/−5%, 10%, 15%, 20%, 25%, 50%, etc. of the values shown. In FIG. 7, the distal tip is an electrode contact formed of platinum/Iridium. Proximal to the distal tip is an insulator sleeve and then a stop configured to limit insertion of the lead into the cochlear scala tympani (e.g., limiting the insertion to a maximum depth of 1 mm, etc.). In this variation, the stop is a silicone rubber stopper circumferentially around the diameter of the lead, extending perpendicularly from the long axis of the lead.

In FIG. 7, the region of the lead proximal to the stop, which is configured to be immediately adjacent to the round window region, includes the insulated wings that are perpendicularly oriented, as shown. The region around this (and slightly distal to it) has an enlarged/reinforced sleeve or surface intended to be manipulated by a surgical tool.

As mentioned, the example lead shown in FIG. 7 is a single channel electrode configured to allow insertion using conventional ENT surgical tools. The single channel electrode contact 701 may be configured to have the same radiating area as a typical large-contact cochlear implant electrode, as known in the art. The insulated sleeve 703 shown in configured for round window membrane pass-through, and may include the neck region shown, so that the tip is held within the cochlear scala tympani region. The silicon stopper 705 may help seal and position the electrode in round window. As mentioned, the reinforced insulated sleeve 707 may be configured for use as a surgical tool contact (“grab”) surface to insert and position the electrode in the round window.

In FIG. 7, the insulated orientation wings 709 may provide additional contact or grab surfaces for surgical placement and to provide a means to adjust rotational orientation of the inserted electrode. The wings in this example are oriented 90 degrees to the plane of the electrode contact tip. The insulated lead 711 shown in FIG. 7 may be made (for example) from platinum, e.g., 50-micron to 100-micron diameter platinum, or MP-35 cardiac pacemaker lead wire.

FIG. 7B shows a front view of the electrode (the radiating /insertion tip of the lead) and the silicone stopper. The silicone stopper may be fabricated from a very soft, low durometer silicone (e.g., silicone of approximately 25 durometer). The lead may also include a two part laminated element with a higher durometer material on the backside of the stopper, enhancing stiffness while protecting the anatomy. The area of the contact in the lead shown in FIG. 7A and 7B is slightly larger than the area of a single ABC HR-90K electrode contact. The point at the distal end (tip) of the electrical contact may be a blunted to prevent metal erosion caused by current crowding and excessive current densities. In some variations, the tip is insulated, or made of a non-conductive material.

Surgical insertion of the lead shown in FIG. 7 may be performed with a small incision to provide reduced insertion resistance and help position the conductive tip at the anterior-inferior corner of the round window. Alternatively, no additional incision may be used.

Leads having more than one channel (e.g., multiple electrical contacts) may also be used. A multi-contact lead may be formed on an elongated region of the distal tip of the device so that the multiple contact regions are separated with an insulating interface positioned between them and behind the tip. The multiple leads may be arranged along the length (longitudinally) or around the circumference (circumferentially) or both.

As mentioned above, the lead shown in FIGS. 7A and 7B may have a higher current concentrated around the sharper edges of the contact. In some variations the lead may be fabricated to avoid such sharp edges. For example, a lead may be fabricated with a ceramic carrier to laminate the metal to each side of the carrier. The leading edge of the ceramic carrier may be sharpened to allow it to puncture the round window membrane without the requirement for an incision and will provide a sharp insertion material unaffected by the dissolution properties of a conductive metal edge.

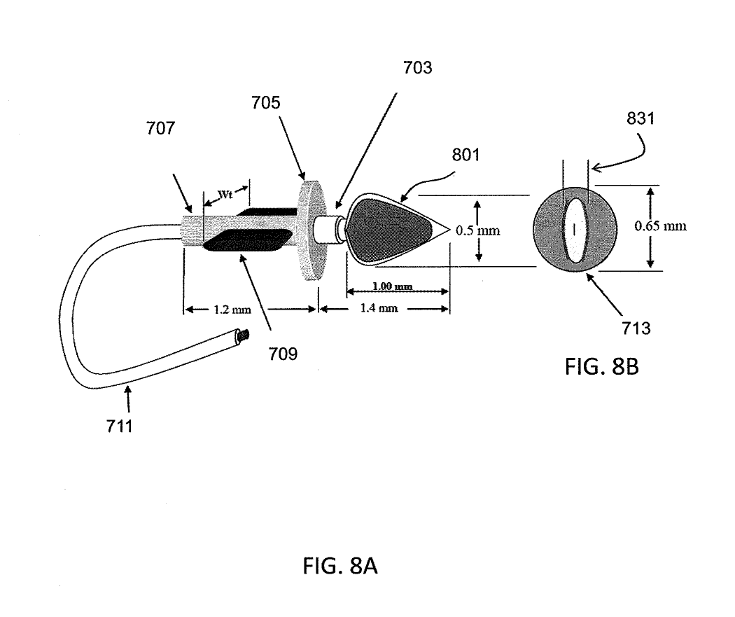

FIG. 8A shows another variation of a lead having a ceramic tip. In this variation, the lead tip 801 is formed as a composite structure having of a non-conductive carrier (ceramic) plated on each side with a conductive material 831 (e.g., platinum/iridium). The carrier material may be non-conductive and able to support a sharp edge. This structure can be fabricated using several methods such as sputtering, electroless plating, lamination, wet metal-plating or any of a number of other proven processes that are compatible with the proposed structure and available biocompatible materials. This structure may provide two (or more) isolated contacts on opposing sides of the carrier. These contacts can be used as either individual channels in a monopolar stimulation mode with a remote return electrode or in a bipolar system where both contacts are used in a differential stimulation mode. Both electrode contacts may also be connected together (e.g., internally) to form a single electrode with a larger surface area. The dimensions of the carrier structure must be increased to provide the surface area necessary for safe stimulation charge densities for high energy stimulation modes.

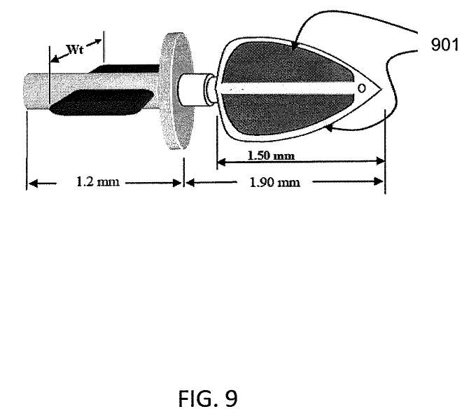

As mentioned, a stabilizing lead as described herein may have any appropriate number of channels (and electrodes or electrical contacts). For example, FIG. 9 shows a variation having four contacts which may be used as a four-channel lead. In this variation, the stabilizing tinnitus lead includes four electrical contacts 901 (electrodes) formed by plating conductive material in two regions of each of the two sides of the distal tip. The dimensions of the distal tip region may be increased to provide sufficient area for each of the four conductive surfaces. The overall fabrication of this example is similar to the above two- contact and one-contact electrode (FIGS. 7A-8B).

Any of the leads described herein may also be configured for drug delivery. For example, the electrode tip may also include a delivery port (or ports) to provide a means to deliver drugs. The lead and connection system could be fitted with a tube to allow for the transport and delivery of drugs that can be used to treat tinnitus. In some variations the lead may include an elutable drug coated or deposited on a portion of the lead (e.g., the tip) to elute a drug into the tissue (e.g., within the perilymph of the cochlear scala tympani).

Although illustrative variations of the present invention have been described above, it will be evident to one skilled in the art that various changes and modifications may be made without departing from the invention.

What is claimed is:

1. A system for treating tinnitus by electrically stimulating the cochlea to supplement the baseline spontaneous neural activity of a subject's cochlea, the system comprising:
an implantable lead configured for insertion through the round window of the cochlea so that one or more electrical contacts at the distal end of the lead is within the cochlear scala tympani to a depth of 1 mm or less;
a signal generator configured to deliver a train of current pulses; and
a controller coupled to the signal generator and configured to modify the train of current pulses from the signal generator so that the current applied by the implanted lead triggers a pattern of cochlear stimulation that is similar to the baseline spontaneous neural activity of a normal cochlea.

2. The system of claim 1, wherein the lead comprises a sharp distal end configured to penetrate the round window of the cochlea.

3. The system of claim 1, wherein the lead comprises a stop located proximally about 1 mm or less from the distal end of the lead.

4. The system of claim 1, wherein the implantable lead comprises a plurality of electrical channels and the system further comprises a multiplexer coupled to the plurality of channels.

5. The system of claim 1, wherein the controller is adjustable to adjust the pattern of current pulses.

6. The system of claim 1, wherein the controller is adjustable by a subject wearing the implantable lead.

7. The system of claim 1, wherein the signal generator is part of an implantable therapeutic stimulator configured to couple with the implanted lead.

8. The system of claim 1, wherein the controller is part of a wearable head-level processor.

9. The system of claim 1, wherein the signal generator forms part of a stimulator including a pulse shape modulator, a burst mode modulator and a dose control modulator, further wherein the controller is configured to control the pulse shape modulator, burst mode modulator and dose control modulator.

10. The system of claim 1, wherein the system does not include a microphone.

11. A system for treating tinnitus by electrically stimulating the cochlea to supplement the baseline spontaneous neural activity of a subject's cochlea, the system comprising:
an implantable lead having one or more electrodes, the lead configured for insertion of the one or more electrodes through the round window into the cochlear scala tympani;
a stimulator configure to apply current to the implantable lead, the stimulator comprising a pulse generator configured to emit a train of current pulses;
a pulse shape modulator configured to modulate the shape of the current pulses emitted by the pulse generator;
a burst mode modulator configured to modulate the emitted train of current pulses to an adjustable burst on-time and burst off-time;
a dose control modulator configured to modulate the emitted train of current pulses to an adjustable dose level; and
a controller configured to control the dose control modulator, burst modulator and pulse shape modulator to emit a pattern of current pulses from the implantable lead that trigger neural activity in a subject's cochlea having a pattern similar to a baseline spontaneous neural activity pattern.

12. The system of claim 11, wherein the lead comprises a sharp distal end configured to penetrate the round window of the cochlea and into the cochlear scala tympani to a depth of 1 mm or less.

13. The system of claim 11, wherein the lead comprises a stop located proximally about 1 mm or less from the distal end of the lead.

14. The system of claim 11, wherein the implantable lead comprises a plurality of electrical channels and the system further comprises a multiplexer coupled to the plurality of channels.

15. The system of claim 11, wherein the controller is adjustable to adjust the pattern of current pulses.

16. The system of claim 11, wherein the controller is adjustable by a subject wearing the implantable lead.

17. The system of claim 11, wherein the signal generator is part of an implantable therapeutic stimulator configured to couple with the implanted lead.

18. The system of claim 11, wherein the controller is part of a wearable head-level processor.

19. The system of claim 11, wherein the system does not include a microphone.

20. A method of treating tinnitus by electrically stimulating the cochlea to mimic the baseline spontaneous neural activity of a subject's cochlea, the method comprising:
inserting an electrical lead within the cochlea; and
applying current pulses within the cochlea from the electrical lead to trigger neural activity that mimics baseline spontaneous neural activity of the subject's cochlea.

21. The method of claim 20, wherein the step of inserting comprises inserting the electrical lead through the round window and into the cochlear scala tympani.

22. The method of claim 20, wherein the step of inserting further comprises implanting the lead so that one or more electrical contacts on the lead extend into the cochlear scala tympani 1 mm or less from the round window of the cochlea.

23. The method of claim 20, wherein the step of applying current pulses comprises applying a train of current pulses between about 3 and 5 kHz.

24. The method of claim 20, further comprising sensing the baseline spontaneous neural activity of the subject's cochlea.

25. The method of claim 20, wherein the step of applying current pulses comprises sensing the spontaneous neural activity of the subject's cochlea and comparing the neural activity to a predetermined target level of baseline spontaneous neural activity.

26. The method of claim 20, wherein the step of applying current pulses comprises allowing the user to adjust the dosage of the applied current pulses.

27. A method of treating tinnitus by electrically stimulating the cochlea to supplement the baseline spontaneous neural activity of a subject's cochlea, the method comprising:
inserting an electrical lead within the cochlea;
sensing the spontaneous neural activity of the subject's cochlea; and
applying current pulses within the cochlea from the electrical lead to supplement the spontaneous neural activity of the subject's cochlea and reduce the tinnitus.

28. The method of claim 27, wherein the step of inserting comprises inserting the electrical lead through the round window and into the cochlear scala tympani.

29. The method of claim 27, wherein the step of inserting further comprises implanting the lead so that one or more electrical contacts on the lead extend into the cochlear scala tympani 1 mm or less from the round window of the cochlea.

30. The method of claim 27, wherein the step of applying current pulses comprises applying a train of current pulses between about 3 and 5 kHz.

31. The method of claim 27, wherein the step of applying current pulses comprises comparing the sensed neural activity to a predetermined target level of spontaneous neural activity.

32. The method of claim 27, wherein the step of applying current pulses comprises allowing the user to adjust the dosage of the applied current pulses.
